(19) United States
(12) Patent Application Publication
(10) Pub. No.: US 2025/0127729 A1
(43) Pub. Date: Apr. 24, 2025

(54) PATCH FOR TARGETED DELIVERY OF AN ONCOGENIC CARGO TO A TISSUE

(71) Applicant: The Board of Trustees of the University of Illinois, Ubana, IL (US)

(72) Inventors: Hyunjoon Kong, Champaign, IL (US); Byoungsoo Kim, Cheongju (KR); Lawrence B. Schook, Champaign, IL (US); Kyle Schachtschneider, Chicago, IL (US); Lobna Elkhadragy, Oak Park, IL (US); Maximillian Carlino, New Haven, CT (US)

(73) Assignee: The Board of Trustees of the University of Illinois, Urbana, IL (US)

(21) Appl. No.: 18/835,464

(22) PCT Filed: Feb. 1, 2023
(86) PCT No.: PCT/US2023/012103

§ 371 Date: Aug. 2, 2024

Related U.S. Application Data
(60) Provisional application No. 63/306,449, filed on Feb. 3, 2022.

(51) Int. Cl. A61K 9/70; A61K 9/00; A61K 48/00

(52) U.S. Cl. CPC A61K 9/703; A61K 9/0024; A61K 48/0008

(57) ABSTRACT

Patches configured to hold an oncogenic cargo and deliver the cargo in a controlled manner to a predetermined location of a specified tissue are disclosed. As one example, a patch can comprise a base layer configured to adhere to a predetermined location of a tissue and a hydrogel layer. The hydrogel layer can comprise a plurality of microchannels that are configured to store and release a gene-modifying vector over a time period and in a quantity sufficient to produce a somatic cell tumor through manipulation of the genome of somatic cells at the predetermined location.

### CROSS REFERENCE TO RELATED APPLICATION

This application claims the benefit of U.S. Provisional Application No. 63/306,449, entitled “Patch for Targeted Delivery of an Oncogenic Cargo to a Tissue,” filed Feb. 3, 2022, which is incorporated by reference herein in its entirety.

### ACKNOWLEDGMENT OF GOVERNMENT SUPPORT

This invention was made with government support under CBET-0939511 awarded by the National Science Foundation. The government has certain rights in the invention.

### FIELD

This disclosure relates to devices that can be in the form of patches used for in vivo delivery of a physiologically active cargo to a tissue.

### BACKGROUND

In the development of medicines and medical devices for use in humans, a non-human animal or “preclinical” development stage must precede the clinical testing stage that is undertaken in human beings. Thus, preclinical development is dependent upon the identification of suitable non-human animals that might model the potential effects of the medicine or medical device before clinical testing can proceed. Identification of suitable and predictive non-human animal models is important for effectively and efficiently bringing new medicines and medical devices to market for the benefit of human health and well-being.

Suitable animal models may assist in assessing clinical feasibility, efficacy, safety, and comparative advantages and disadvantages of a new medicine or medical device prior to or as an adjunct to clinical testing on human subjects. Goals of preclinical studies for new medicines can be to determine a starting, appropriate dose for first-in-human studies and/or assess the potential toxicity of the product. For medical devices, preclinical testing models can assess the practicality of the device and potential limitations on the use of the device. Further, for medical diagnostics, preclinical models can provide a platform for testing or studying liquid biopsy assays or various imaging modalities.

As an example, preclinical models (e.g., non-human animal models) for certain disease states, such as cancer, can be used to study oncolytic medicines and medical devices. Well-established preclinical studies typically rely on small laboratory animal models (e.g., mice, rats, and rabbits), notwithstanding the many drawbacks of such animal models. These small laboratory animal models can be a poor predictor of a medicine's or medical device's performance and/or toxicity in humans due to substantial differences from humans in anatomy, physiology, genetics, and metabolism.

Some more recently developing preclinical models (e.g., notably porcine models) have higher homology to humans and similar tumor phenotypes. Such models have been shown to be better predictors of both safety and efficacy for human medicines and medical devices. In developing preclinical models for the treatment of human diseases, such as the various human cancers, a significant challenge has been the lack of so-called autochthonous animal models in which the animal model is able recapitulate the clinical oncogenic process of tumor initiation at the site of tumor formation in a specific tissue. Absent autochthonous animal models, preclinical investigation of oncolytic medicines and medical devices can depend upon use of transplantation models in which a tumor-containing cells are transplanted into a test animal in order to study the effects of a medicine or medical device that might be useful in the treatment of the tumor in question.

Thus, a need exists for creating autochthonous tumors in preclinical animal models in a more reliable and more efficient manner such that preclinical studies of medicines and medical devices might proceed with a higher degree of predictability of possible clinical outcomes form the use of such medicines and devices.

### SUMMARY

Disclosed herein are devices and methods for their use that are capable of the targeted, in vivo delivery of a physiologically active cargo to the tissue of an animal (such as oncogenic, gene-modifying vectors). The devices disclosed herein include an oncogenic cargo-containing matrix that can be configured in the form of a patch holding the oncogenic cargo prior to its delivery to a tissue, e.g., over a period of time that may be predetermined. Thus, the devices disclosed herein include oncogenic patches that can be placed in contact with the selected tissue in vivo, e.g., at a predetermined location, in order for the device to then deliver the oncogenic cargo. In this regard, the device may be implanted surgically (or less invasively, such as through an endoscope or needle) to make contact with the tissue selected for the delivery of the oncogenic cargo at a predetermined site. More specifically, patches are disclosed that comprise a hydrogel with a plurality of microchannels configured to hold the oncogenic cargo prior to its delivery to the selected tissue. In particular, oncogenic patches are disclosed that comprise an additional layer capable of adhering the patch to a selected tissue, e.g., a polydopamine adhesive layer. The hydrogel patches containing the physiologically active cargo in the form of oncogenic, gene-modifying vectors may deliver carcinogens, viral vectors, gene editing components, or combinations thereof that can induce tumor growth in cells of the selected tissue. In this way, the patches and methods described here can be utilized to initiate autochthonous tumor formation and subsequently induce tumor growth at a selected site of a targeted tissue of an intact animal. The construction and implantation of the cargo-carrying device thereby provides a preclinical animal model for tissue- and organ-specific tumor formation that can be used to predict the potential safety, efficacy, advantages, limitations, or other effects of medicines and devices of possible clinical value in human subjects.

In one example, a patch can include a base layer configured to adhere to a predetermined location of a tissue and a hydrogel layer comprising a plurality of microchannels that are configured to store and release a gene-modifying vector over a time period and in a quantity sufficient to produce a somatic cell tumor through manipulation of the genome of somatic cells at the predetermined location.

In another example, a method for the production of a somatic-cell tumor at a predetermined location of a tissue can include positioning, at the predetermined location, a patch comprising a base layer configured to adhere to the predetermined location and a hydrogel layer comprising a plurality of microchannels containing a releasable oncogenic cargo stored within the plurality of microchannels, such that the oncogenic cargo is released from the hydrogel layer and produces a somatic-cell tumor at the predetermined location through manipulation of the genome of the somatic cells.

In another example, a method of making a microchannel-containing hydrogel patch can include forming a hydrogel by cross-linking gel-forming molecules in a cross-linking solution, adhering a base layer on a surface of the hydrogel, the base layer configured to adhere to a tissue, lyophilizing the hydrogel under conditions sufficient to generate a plurality of microchannels such that a cryogel with the plurality of microchannels is formed, and rehydrating the cryogel in a solution containing a selected cargo.

In another example, a method of making a microchannel-containing hydrogel can include mixing a flexible cross-linked hydrogel with a solution comprising a cargo, lyophilizing the flexible cross-linked hydrogel and cargo under conditions sufficient to generate a plurality of microchannels comprising the cargo in the hydrogel, and adhering a base layer on a surface of the hydrogel, the base layer configured to adhere to a tissue.

The foregoing and other features of the disclosure will become more apparent from the following detailed description, which proceeds with reference to the accompanying figures.

### BRIEF DESCRIPTION OF THE DRAWINGS

FIG. 1A is a schematic of an exemplary patch configured to hold and controllably release a cargo.

FIG. 1B is a schematic of the exemplary patch of FIG. 1A being positioned at a tissue.

FIG. 2 is a schematic of an exemplary patch comprising a flexible cross-linked hydrogel and a polydopamine base layer.

FIG. 3A shows an exemplary patch comprising an alginate and polydopamine hydrogel with a plurality of microchannels or micropores configured to be loaded with a specified cargo.

FIG. 3B is a microscopic image of a portion of the patch of FIG. 3A showing micropores in the patch.

FIG. 4A shows the patch of FIG. 3A in a folded state.

FIG. 4B shows the patch of FIG. 3A in an unfolded state.

FIG. 5 show the patch of FIG. 3A adhered to a tissue via its polydopamine base layer.

FIG. 6 is a flow chart of a method for making a patch comprising a flexible cross-linked hydrogel with a plurality of microchannels and a polydopamine base layer, the microchannels configured to hold a selected cargo.

FIG. 7 is a flow chart of another method for making a patch comprising a flexible cross-linked hydrogel with a plurality of microchannels and a polydopamine base layer, the microchannels configured to hold a selected cargo.

FIG. 8A is a front view of an exemplary patch configured as a disc.

FIG. 8B is a side view of the patch of FIG. 8A.

FIG. 9 shows the patch of FIG. 8A in various stages of unfolding, thereby demonstrating its ability to be transformed into a tubular shape and then undergo unrolling.

FIGS. 10A-10C are schematics depicting an exemplary method for delivering a patch to a target tissue using an endoscope.

FIG. 11 is a flow chart of an exemplary method for inducing formation of a tumor at a predetermined location of a tissue using a patch.

FIG. 12 presents microscopic images of three differently sized patches and green fluorescent protein (GFP) expression in each of the patches following placement at the bladder epithelium.

FIG. 13 presents a brightfield image of a tissue exposed to AdCre via a patch and a control patch, as well as an image showing AdCre-GFP expression in the tissue exposed to the patch and the control.

FIG. 14 presents microscopic images that show tumor formation around implanted patches as compared to a control.

FIG. 15 presents microscopic images of GFP expression in a tissue exposed to a patch with AdCre and a control patch with GFP only.

FIGS. 16A-16F are a series of panels illustrating development of Oncopig lung adenocarcinoma cell lines. FIG. 16A is an image of primary Oncopig type II pneumocytes. FIG. 16B is an image showing that treatment of the Oncopig type II pneumocytes with Ad-Cre results in vacuolization and cell death. FIG. 16C is an image showing positive TTF1 staining of Oncopig lung adenocarcinoma cell lines. FIG. 16D is a gross image of ex vivo lung nodule. FIG. 16E is an image showing GFP expression in ex vivo lung nodule. FIG. 16F is a histological image displaying architecture consistent with lung adenocarcinoma.

FIGS. 17A-17C are a series of panels illustrating development of rabbit HCC cell lines. FIG. 17A is an image showing primary rabbit hepatocytes. FIG. 17B is a graph showing editing percentage for rabbit hepatocytes 3 weeks pos-CRISPR-Cas9 editing. FIG. 17C is an image showing positive arginase-1 staining of rabbit TP53 and PTEN knockout cell lines, confirming hepatocellular origin.

### DETAILED DESCRIPTION

Disclosed herein are devices configured to deliver an oncogenic cargo to a selected tissue and methods for using such devices as a preclinical, in vivo model of predictive relevance to the possible clinical effects or outcomes of the use medicines, diagnostics, and devices in diagnosing and treating cancers in humans. In some examples, the device is an oncogenic patch that comprises a flexible cross-linked hydrogel comprising a plurality of microchannels and a base layer attached to one side of the hydrogel and configured to adhere to a tissue, e.g., at a predetermined location of the selected tissue. This configuration of the patch permits it to hold a cargo within the microchannels. Once adhered to a tissue, the cargo can be controllably released (e.g., at a predetermined diffusion rate out of the oncogenic patch for delivery to the cells of the tissue, over a period of time).

In some examples, the oncogenic patches can be flexible and can be at least partially folded or rolled to be inserted into a delivery apparatus and delivered in a relatively non-invasive manner to the target tissue. In some examples, the delivery apparatus can be an endoscope or another delivery catheter.

In some examples, the cargo can be a cancer causing cargo that can induce tumor formation, growth, or both, thereby resulting in a selected cancer to manifest in a subject. The subject can, in some examples, comprise an animal that is to be used as a preclinical model (such as a large animal model, for example a pig) for testing various anticancer medicines, medical devices, or diagnostic techniques. In certain examples, the patches and methods described herein can be tailored to effectively and efficiently create autochthonous tumors in preclinical animal models.

### Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various examples of the disclosure, the following explanations of specific terms are provided:

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrated to other parts of the body, for example via the bloodstream or lymph system.

Cargo: A substance or substances, such as viral vectors, genetic modifying components, oncogenic nanoparticles, carcinogens, or other substances that can be included or incorporated into a patch (e.g., within microchannels of the patch) and which can create or treat disease at the implantation site (such as a tissue). The cargo can be an oncogenic cargo, i.e., a cargo comprising a physiologically active substance or substances that induce tumor formation or tumor growth, or both, in a tissue to which the cargo is delivered.

Oncogenic: A molecule, material, or device that causes the development of a tumor or tumors.

Oncopig: An inducible porcine cancer model, referred to as the “Oncopig” or “Oncopig Cancer Model” (OCM). This porcine model of cancer is a transgenic porcine line encoding Cre recombinase-inducible porcine transgenes encoding KRAS^{G12D} and TP53^{R167H} (see, e.g., Schook et al., PLOS One 10: e0128864, 2015). Cells from the Oncopig can be transformed in culture with an adenovirus encoding Cre (AdCre). Furthermore, injection of the transgenic pigs with AdCre results in formation of tumors.

Pharmaceutically acceptable carrier: Remington: The Science and Practice of Pharmacy, 22^{nd} ed., London, UK: Pharmaceutical Press (2013), describes compositions and formulations suitable for pharmaceutical delivery of cargoes or other compositions disclosed herein. In general, the nature of the carrier will depend on the particular mode of administration being employed. For instance, formulations for inclusion in a patch described herein can include pharmaceutically and physiologically acceptable fluids such as water, physiological saline, balanced salt solutions, aqueous dextrose, glycerol or the like as a vehicle. In addition to biologically neutral carriers, compositions to be administered or included in a patch can contain minor amounts of non-toxic auxiliary substances, such as wetting or emulsifying agents, preservatives, and pH buffering agents and the like, for example sodium acetate or sorbitan monolaurate.

Subject: A living multi-cellular vertebrate organism, a category that includes human, veterinary, and laboratory subjects, including human and non-human mammals (such as mice, rats, rabbits, and pigs).

Tumor: The product of neoplasia is a neoplasm (a tumor), which is an abnormal growth of tissue that results from excessive cell division. A tumor that does not metastasize is referred to as “benign.” A tumor that invades the surrounding tissue and/or can metastasize is referred to as “malignant” or “cancer.” Neoplasia is one example of a proliferative disorder. An “autochthonous” tumor is one that is initiated in normal cells and in a whole organism (such as a mouse or pig) and is considered to more closely mimic human tumors than models utilizing implanted or injected tumors or tumor cells (such as xenografts).

Patch: A hydrogel configured to adhere to a tissue and carry a cargo within its interior. The patch is configured to be delivered to a targeted location of a selected tissue and then, upon contact with the tissue, release its cargo over a predetermined period of time. An “oncogenic patch” references a patch containing an oncogenic cargo.

Vector: A nucleic acid molecule that can be introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function.

### Description of a Patch Device

As introduced above, there is a need for preclinical models for various disease states, such as cancers, that are relatively inexpensive, amenable to high through-put screening, and closely resemble the disease state as it would be presented in humans. As an example, it is desirable to recapitulate the complexity of specified human tumors within a preclinical in vivo (e.g., animal) model. While directly transplanting or injecting cancer cells into selected preclinical animal models can result in tumor formation, such methods can result in the inability to precisely control the site of tumor formation within a target tissue and/or an uncontrollably fast disease course. As a result, the applicability of such models for preclinical medicine, medical device, and diagnostic testing can be limited.

The issue of limited control of tumor location and size within a preclinical model may be especially problematic for preclinical models of luminal cancers, such as bladder, esophagus, stomach, pancreatic, and colorectal cancers.

Thus, the inventors herein have recognized that minimally invasive delivery approaches that allow for more controlled localization and release of tumor inducing compositions may trigger the formation of various cancers (such as cancers of the luminal organs listed above) in a more controlled, defined location. In addition, the approaches provided herein can also be applied to non-luminal organs or other tissue types.

A patch configured to carry a cargo (which in some examples can comprise tumor-inducing materials or particles, as described further herein) can be configured to adhere to a target tissue and provide controlled, sustained delivery of the cargo at the target tissue. In some examples, the delivered cargo can induce formation or growth of a tumor in the target tissue (e.g., at or close to the site of the oncogenic patch). In this way, the patch with its cargo can induce tumor formation or growth at a defined location and growth rate in the target tissue in vivo (e.g., in an animal model). The patch can be further configured to degrade after delivery of the cargo at the target tissue. In some examples, such patches can be flexible such that they may be delivered endoscopically or via another minimally invasive targeted delivery method. As described further herein, this type of delivery approach can allow for the patch delivery procedures to be performed using existing veterinary, biomedical, and clinical facilities and techniques.

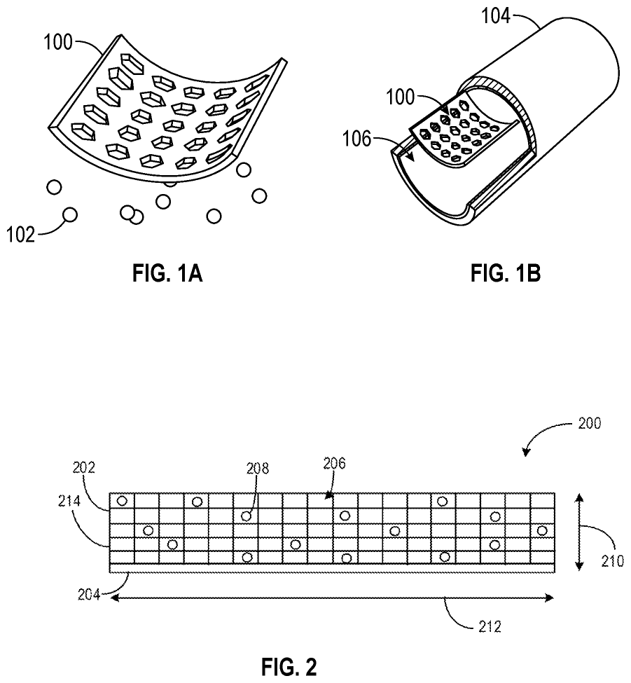

FIGS. 1A and 1B show an exemplary patch 100 configured to hold and controllably release a cargo 102 (FIG. 1A) and adhere to a tissue 104 (FIG. 1B). The patch 100 can also be referred to as a tumor inducing patch (TIP) or a tumor modulating patch (TMP) (e.g., when the cargo 102 comprises oncogenic viral vectors, oncogenic nanoparticles, and/or carcinogens that can induce tumor formation or growth). In some examples, the patch 100 can comprise a flexible, cross-linked hydrogel that is configured to store the cargo 102 (within microchannels or micropores formed in the hydrogel, as described further below with reference to FIG. 2) and then controllably release the cargo 102 (FIG. 1A) after implantation at the tissue 104 over a predetermined period of time.

In some examples, as shown in FIG. 1B, the patch 100 can be delivered to an inner lumen 106 (e.g., an inner luminal surface) of the tissue 104. For example, the tissue 104 can be part of a luminal organ, such as the bladder, esophagus, colon, nerves, and the like. In other examples, the tissue 104 can be part of a solid organ, such as a lung, liver, heart, kidney, bone, and the like. In some examples, the tissue 104 can be an exterior surface of a tissue of an organ, such as an ovary, neuron, or brain. Thus, the patch 100 can be flexible and configured to bend and conform to a curved inner lumen 106 of the tissue 104 (or a curved, irregular, or convex outer surface of a solid organ).

FIG. 2 is a schematic of an exemplary patch 200, which may be similar to the patch 100 of FIGS. 1A and 1B and can be used in the same way as described above with reference to FIGS. 1A and 1B. As introduced above, the patch 200 can comprise a flexible cross-linked hydrogel 202. The hydrogel 202 can comprise gel-forming biocompatible molecules. In some examples, the hydrogel 202 is an alginate and divalent hydrazide group cross-linked hydrogel. In other examples, the hydrogel 202 can comprise alginate, hyaluronic acid, chitosan and their derivatives, poly (ethylene glycol) (PEG) diacrylates, PEG dimethacrylates, collagen, gelatin, gelatin methacrylates, poly (lactic-co-glycolic acid), or combinations thereof.

The hydrogel 202 can be configured to degrade after contact with a tissue (e.g., tissue 104 of FIG. 1B) over a predetermined time period. In some examples, the patch 200 can degrade at a rate that supports tumor growth without impeding the environment for tumor growth, and while also reducing or avoiding a foreign body reaction (such as an undesired immune response to the patch). In some examples, this predetermined time period is in a range of three to five weeks. For example, the hydrogel 202 can comprise one or more biodegradable materials. The predetermined time period for degradation of the patch 200 can by adjusted by adjusting the composition of the hydrogel 202 (e.g., by selecting from one or more of the gel-forming molecules listed above to achieve the predetermined time period for degradation).

The hydrogel 202 of the patch 200 can comprise a plurality of pores or microchannels 206 that are configured to receive cargo 208 therein. For example, the patch 200 can be configured to contain the cargo 208 within lumina of the plurality of microchannels 206 (as shown in FIG. 2) and, upon contact with a tissue, continually release the cargo from the microchannels 206 over a predetermined time period (which can be different than the predetermined time period for degradation of the hydrogel 202). In this way, the microchannels 206 can be configured such that the cargo 208 slowly diffuses out of the microchannels 206 over the predetermined time period and/or at a set diffusion rate.

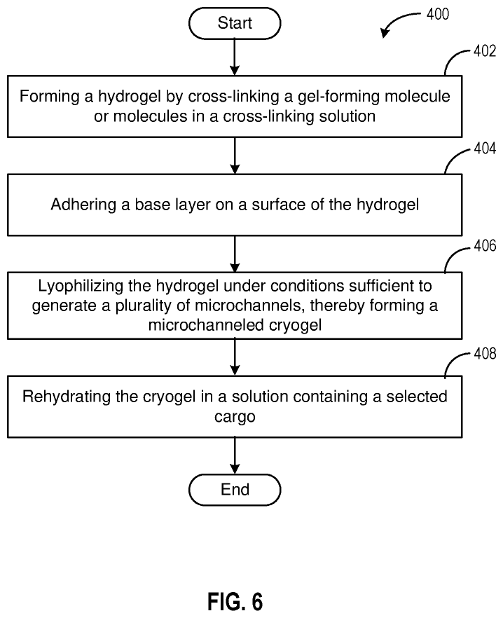

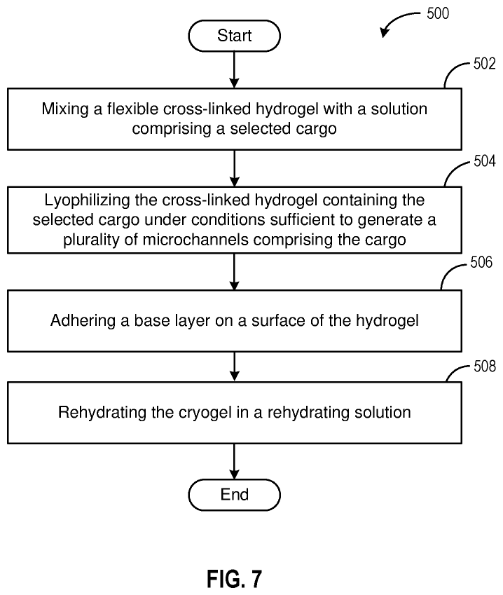

In some examples the plurality of microchannels 206 can be anisotropically aligned with one another in the hydrogel 202. Further, in some examples, the microchannels 206 (or pores) can have an opening width or diameter in a range of 50-1000 μm (such as 50-100 μm, 100-300 μm, 200-400 μm, 300-500 μm, 400-600 μm, 500-700 μm, 600-800 μm, 700-900 μm, or 800-1000 μm). In one example, the opening width or diameter is in the range of 200-300 μm. For example, though the microchannels 206 are shown as having a rectangular cross-section in FIG. 2 (for ease of illustration), in other examples, the microchannels 206 can have a circular or oblong cross-section having a diameter or major diameter in the range of 50-1000 μm. The width or diameter of the microchannels 206 can be selected to achieve a desired diffusion rate or speed of release of the cargo 208 from the microchannels 206. For example, smaller pores or diameters of the microchannels 206 can result in slower diffusion rates and a longer time period for complete release of the cargo 208. In contrast, as the pore size or diameter of the microchannels 206 increases, the diffusion rate for the cargo can decrease. Formation of the microchannels 206 within the hydrogel 202 is described further below with reference to FIGS. 6 and 7.

Additionally, in some examples, by modulating a tortuosity, charge density, pore size, or combinations thereof, of the microchannels 206, varying diffusion rates or speed of release of the cargo 208 from the microchannels 206 can be achieved. In some examples, multiple patch layers of the hydrogel 202 can be 3D printed with various pore sizes and compositions to allow for release of various cargos at different rates or time points. Further, in some examples, cargo-containing microparticles can be included within the patch 200 which allow for slower release of the cargo. Thus, using these approaches, multiple cargos can be loaded into the patch 200 and delivered at various rates.

In some examples, a portion of the patch 200 containing the microchannels 206 can be referred to herein as a cargo layer 214 of the patch 200.

The patch 200 can further comprise a base layer 204 configured to adhere to a tissue. In some examples, as shown in FIG. 2, the base layer 204 can be attached to one side of the hydrogel 202 and configured to adhere to a tissue (e.g., tissue 104 of FIG. 1B). The base layer 204 can act as glue to enable the patch 200 to adhere to the tissue (such as an inner lumen lining of the tissue, as shown in FIG. 1B) until it has delivered its cargo and then dissolves or degrades. The base layer 204 can comprise fibrin or polydopamine (e.g., a fibrin or polydopamine-based tissue glue or glue layer).

In some examples, the patch 200 can have a thickness 210 in a range of 0.05-5 mm, a length 212 in a range of 1-10 mm, and a width (a direction that is perpendicular to the length 212 and thickness 210 and shown into the page in FIG. 2) in a range of 1-10 mm. Dimensions of several exemplary patches are presented in Table 1, as described further below. The dimensions of the patch 200 can be selected and varied based on the predetermined location at the target organ for implantation. In some examples, the dimensions of the patch 200 can be additionally or alternatively selected based on a specified tumor size and growth rate.

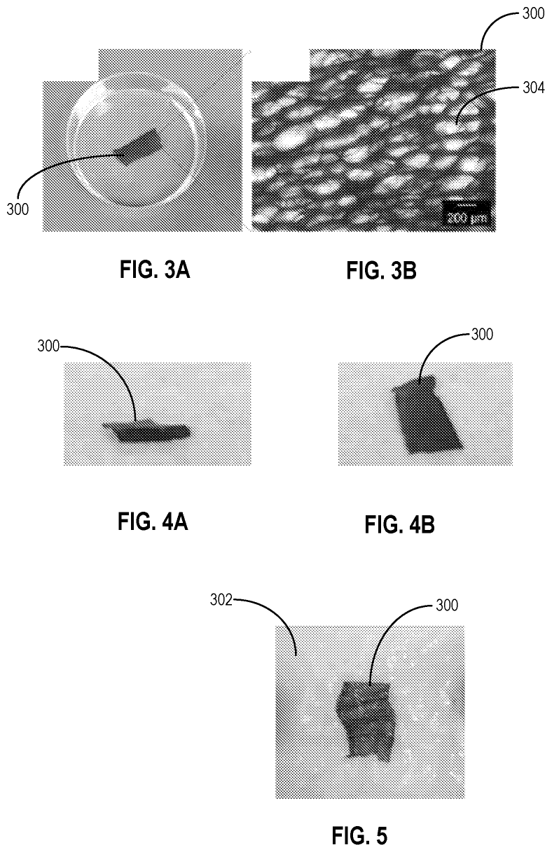

FIGS. 3A-5 show images of an exemplary patch 300 configured to be loaded with a selected cargo (e.g., a cancer-causing or tumor-inducing cargo, as described further below) and release the cargo following adherence of the patch 300 to a tissue 302 (FIG. 5). In some examples, the patch 300 with its cargo can be configured to support the timely (e.g., within a predetermined time period) development of site and cell-specific solid tumors representative of clinically relevant size and biology for a specified animal model (e.g., pig or other large animal).

FIG. 3A shows the exemplary patch 300 comprising an alginate and polydopamine hydrogel while FIG. 3B is a microscopic image of a portion of the patch 300 showing micropores 304 in the patch 300. In alternate examples, the patch 300 can comprise microchannels in lieu of the micropores 304, wherein the microchannels are configured as channels, which typically have open ends, and that can be anisotropic ally aligned within one another within the patch 300.

FIG. 4A shows the patch 300 in a folded state or configuration while FIG. 4B shows the patch 300 in an unfolded state or configuration. The patch 300 can be folded and unfolded repeatedly, as necessary (e.g., during storage and/or delivery to the tissue 302) due to is flexible hydrogel structure and other geometrical characteristics described herein (e.g., its thickness).

As shown in FIG. 5, the patch 300 can comprise a base layer (e.g., base layer 204 of FIG. 2) that allows it to adhere (e.g., stick and remain stuck) to the tissue 302. The patch 300 can be configured to remain adhered to the tissue 302 even under stretching or pulling of the patch 300 (e.g., due to movement of the organ in which the tissue 302 is part of). Further, the patch 300 can be configured to remain adhered to the tissue 302 until it has delivered its cargo and degrades. Following implantation of the patch 300 and adherence to the tissue 302, as shown in FIG. 5, the patch 300 can release the cargo disposed within the microchannels 304 controllably and locally via the microchannels 304 and degrade over a predetermined time period without causing host inflammation.

The patches described herein can be constructed by forming a cross-linked hydrogel and lyophilizing the cross-linked hydrogel under conditions sufficient to generate a plurality of microchannels. The cargo to be delivered to a tissue via the patch can either be introduced into the hydrogel through a rehydration process, after formation of the microchannels (FIG. 6) or by mixing the cargo with one or more components of the hydrogel before or after the cross-linking (FIG. 7).

As one example, FIG. 6 presents a method 400 for making a patch, such as any one of the patches described herein. The method 400 begins at 402 by forming a hydrogel by cross-linking a gel-forming molecule or molecules in a cross-linking solution. As introduced above, the gel-forming molecule or molecules can include alginate, hyaluronic acid, chitosan and their derivatives, poly (ethylene glycol) (PEG) diacrylates, PEG dimethacrylates, collagen, gelatin, gelatin methacrylates, poly (lactic-co-glycolic acid), or combinations thereof. As one specific example, forming the alginate-based hydrogel at 402 can include cross-linking uronic acid of alginate and hydrolytically labile molecules with divalent hydrazide groups.

The method 400 continues to 404 and includes adhering a base layer (e.g., the base layer 204 of FIG. 2 which can comprise fibrin or polydopamine in some examples) to a surface of the formed hydrogel. A mixture of fibrinogen and thrombin can create a fibrin layer on the base layer of the hydrogel. The fibrinogen and thrombin concentrations can vary to tune the adhesion strength of the fibrin base layer. In other examples, a polydopamine glue base layer can be created by polymerizing dopamine in an oxidative environment. In other examples, polydopamine and fibrin can be hybridized together into a base layer to increase the toughness and adhesion strength of the base layer. In some examples, the method at 404 can occur following the method at 406, as described below.

The method at 406 includes lyophilizing the hydrogel under conditions that are sufficient to generate a plurality of microchannels. For example, the lyophilizing at 406 can include placing the hydrogel on a metal (e.g., copper) substrate at sub-zero temperature to induce uniaxially aligned ice crystals to form through the hydrogel, followed by lyophilization (e.g., freeze-drying and/or dehydrating). This uniaxial freezing process can induce anisotropic ice crystal growth in the hydrogel, while excluding cross-linked polymer chains from the ice crystals. The following lyophilization removes the ice crystals, thus resulting in a cryogel with anisotropically aligned microchannels.

In some examples, the uniaxial freezing and lyophilization of the hydrogel at 406 can result in anisotropically aligned microchannels (e.g., microchannels 206 shown in FIG. 2) with a diameter in a range of 50-1000 μm. Such microchanneled structure enables loading of cargo into the hydrogel (or cryogel) quickly through a rehydration process. The method at 408 can include rehydrating the cryogel (e.g., the dehydrated hydrogel) in a solution containing the selected cargo. In some examples, the solution can comprise deionized water and/or biocompatible buffers or other pharmaceutically acceptable carriers, along with a selected cargo. As described further below, in some examples the cargo can include gene-modifying vectors or oncogenic molecules, such as oncogenic viral vectors, gene editing components, oncogenic nanoparticles, carcinogens, or combinations thereof configured to induce tumor formation or growth.

In some examples, the method 400 can end at 406 and the dehydrated patch (which can be sterile) can be stored without a loaded cargo for a period of time prior to use (for example, up to about one year at room temperature and up to about three years in a frozen state). When ready for use, the dehydrated hydrogel can be rehydrated with the cargo-containing solution, as described above with reference to the method at 408.

In some examples, the dehydrated patch and a selected cargo containing solution can be kept separate and included within a kit. In this way, dehydrated patches can be used for a variety of applications (e.g., different tumor or cancer models, for therapeutic applications, and the like) by selecting a cargo-containing solution from a plurality of available cargo-containing solutions. In this way, a kit for treating a disease or creating a preclinical cancer model can comprise a dehydrated patch (as described above) and a selected cargo-containing solution. In other examples, a dehydrated patch can be combined with a user-selected cargo-containing solution for customized use.

As another example, FIG. 7 presents a method 500 for making a patch, such as any one of the patches described herein. The method 500 begins at 502 by mixing a flexible cross-linked hydrogel with a solution comprising a cargo. In some examples, mixing the cross-linked hydrogel with the solution comprising the cargo at 502 can include mixing alginate or another gel-forming molecule or molecules with the selected cargo (alone or in a solution) and a cross-linking solution (such as described above at 402 of method 400) to form a cross-linked hydrogel containing the selected cargo.

The method 500 continues to 504, which includes lyophilizing the flexible cross-linked hydrogel containing the selected cargo under conditions sufficient to generate a plurality of microchannels comprising the cargo. The method at 504 can be similar to the method at 406, as described above with reference to FIG. 6.

At 506, the method includes adhering a base layer on a surface of the hydrogel (similar to as described above for the method at 404 in FIG. 6).

The method 500 can then continue to 508 to rehydrate the cryogel formed via the methods at 504 and 506 with a rehydrating solution. In some examples, the rehydrating solution can comprise deionized water and/or biocompatible buffers or other pharmaceutically acceptable carriers.

In this way, in some examples, the methods 400 and 500 of FIGS. 6 and 7, respectively, can be used to form a patch configured to adhere to a surface of a selected tissue (such as a luminal surface of a luminal organ or an outer surface of a solid organ) and provide controlled, sustained delivery of a selected cargo to precise locations within or on a tissue (e.g., a predetermined location, such as a luminal structure), thereby resulting in the induction of autochthonous carcinomas with defined locations and growth rates in target tissues in vivo. Thus, animal models of selected disease states (e.g., cancers) can be created for preclinical studies. In other examples, the patch is configured to provide controlled, sustained delivery of a selected therapeutic or candidate therapeutic compound.

In other examples, the patches disclosed herein can be formed by 3D printing the hydrogel (or cargo layer) and then loading the 3D printed hydrogel with the cargo, as described above. As described herein, 3D printing can allow for the formation of multiple patch layers with varying pore size (or microchannel diameter) and compositions, thereby allowing for multiple cargos to be released from the multi-layer patch at different rates or time points.

### Cargo.

The cargo loaded into and contained within the patches described herein can be an oncogenic (e.g., a tumor or cancer-causing) cargo comprising one or more oncogenic viral vectors, genetic modifying components, recombinant proteins, carcinogens, or combinations thereof that can induce formation or growth of a selected tumor. In some examples, the cargo is a gene-modifying vector or agent and can, in some examples, be capable of manipulating the genome of the somatic cells of the tissue at which the patch is positioned. In some examples, the cargo is included in nanoparticles (e.g., oncogenic nanoparticles or nanoparticles housing oncogenic agents) for delivery.

As one example, the cargo can be a viral vector that includes one or more nucleic acids that can induce formation or growth of a tumor (e.g., an oncogenic viral vector). In some examples, the subject to which the patch will be administered or applied is a transgenic animal (such as a mouse or pig) that includes one or more inducible genes that can result in tumor formation or growth when expressed in the animal. For example, the animal may be transgenic for one or more Cre-inducible nucleic acids that can induce tumor formation or growth, such as one or more transgenes including cancer driver mutations. Thus, in some examples, the cargo is a viral vector (such as an adenoviral vector, adeno-associated viral vector, or lentiviral vector) that includes a nucleic acid encoding the Cre recombinase. Expression of the Cre recombinase at or near a location including cells including Cre-inducible nucleic acids results in expression of the inducible nucleic acid(s) and formation or growth of a tumor, if the inducible nucleic acid(s) are oncogenic. One example is the “oncopig” porcine line which is transgenic for a nucleic acid encoding Cre recombinase-inducible porcine transgenes encoding KRAS^{G12D} and TP53^{R167H} (see, e.g., Schook et al., PLOS One 10:e0128864, 2015). Expression of Cre, for example, from an AdCre vector, results in formation of tumors in the oncopig.

In other examples, the cargo is a vector (such as an expression vector) encoding an oncogenic nucleic acid (such as a nucleic acid encoding a driver mutation).

In other examples, the cargo includes gene modifying components that can induce tumor formation or growth upon release from the oncogenic patch by editing the genome, such as by specific nucleotide changes, and additions or deletions ranging from single nucleotide to entire genes, or by inducing certain gene expression. Gene modifying cargo can include recombination proteins, oncogenic expression vectors, gene expression vectors, gene editing components, epigenetic modifiers, ageing modifiers, or combinations thereof.

Exemplary gene editing technologies include those based on genome editing proteins, such as zinc finger nucleases, TALENs, and CRISPR systems. In particular examples, the gene editing is a CRISPR (Clustered Regularly Interspaced Short Palindromic Repeats)/Cas (CRISPR-associated) system. CRISPR systems use RNA-guided nucleases termed CRISPR-associated or “Cas” endonucleases (e.g., Cas9 or Cpf1) to cleave DNA. In a typical CRISPR/Cas system, a Cas endonuclease is directed to a target nucleotide sequence (e.g., a site in the genome that is to be sequence-edited) by sequence-specific, non-coding guide RNAs that target single- or double-stranded DNA sequences. One type of CRISPR system includes a type II Cas endonuclease such as Cas9, a CRISPR RNA (crRNA), and a trans-activating crRNA (tracrRNA). The Cas9 crRNA contains a spacer sequence, typically an RNA sequence of about 20 nucleotides (in various examples this is 20, 21, 22, 23, 24, 25, or up to about 30 contiguous nucleotides in length) that corresponds to (e.g., is identical or nearly identical to, or alternatively is complementary or nearly complementary to) a target DNA sequence of about equivalent length. The Cas9 crRNA also contains a region that binds to the Cas9 tracrRNA to form a partially double-stranded structure which is cleaved by RNase III, resulting in a crRNA: tracrRNA hybrid or duplex. The crRNA: tracrRNA hybrid then directs the Cas9 endonuclease to recognize and cleave the target DNA sequence. In some examples, a tracrRNA and crRNA (e.g., a crRNA including a spacer sequence) can be included in a chimeric nucleic acid referred to as a single guide RNA (sgRNA).

Other CRISPR nucleases useful in methods and compositions of the disclosure include Cpf1, C2c1 (also known as Cas12b) and C2c3 (also known as Cas12c) (see Shmakov et al. (2015) Mol. Cell, 60:385-397). For the purposes of gene editing, CRISPR arrays can be designed to contain one or multiple guide RNAs (including spacer sequences) corresponding to a desired target DNA sequence; see, for example, Cong et al. (2013) Science, 339:819-823; Ran et al. (2013) Nature Protocols, 8:2281-2308. The design of effective guide RNAs for use in plant genome editing is disclosed in US Patent Application Publication 2015/0082478 A1, the entire specification of which is incorporated herein by reference. More recently, efficient Cas9 gene editing has been achieved using a chimeric “single guide RNA” (“sgRNA”), an engineered (synthetic) single RNA molecule that mimics a naturally occurring crRNA-tracrRNA complex and contains both a tracrRNA sequence (for binding the Cas9 nuclease) and at least one crRNA sequence (a guide RNA sequence including a spacer sequence to guide the Cas9 nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in Cas9 genome editing; see, for example, Hendel et al. (2015) Nature Biotechnol., 33:985-991.

In one example, the cargo includes CRISPR gene editing components, which may include nucleic acids or ribonucleoprotein complexes. For example, the cargo may be nucleic acids designed to introduce one or more mutations in a cancer driver gene or knockout expression of a tumor suppressor, as well as a nucleic acid encoding a CRISPR endonuclease (such as appropriate guide RNAs and Cas9). In other examples, the CRISPR gene editing components are provided in the form of a ribonucleoprotein complex including gene editing nucleic acids and an endonuclease protein.

In other examples, the cargo includes synthetic vectors, or materials that bind electrostatically to DNA or RNA, thereby condensing the genetic material into nanometer-scale complexes (e.g., a few tens to several hundred nanometers in diameter) that protect the genes and allow them to enter cells. Such materials can include cationic peptides, proteins, polymers, and liposomes. Exemplary synthetic vectors used for in vitro gene transfer include (diethylamino) ether (DEAE)-dextran and calcium phosphate.

In yet another example, the patch can include one or more auxiliary or additional cargos that can further modulate tumor biology, the tumor microenvironment, and tumor growth rates. Such additional cargo can include cell-division factors, angiogenic factors, carcinogens or combinations thereof. These factors can include stem cell and growth factors to increase cell division and tumor growth rates, angiogenic factors to increase neoangiogenesis, immune modulators to alter the tumor immune microenvironment, and carcinogens to further promote tumor development and heterogeneity.

In other examples, the cargo is a carcinogen. For example, the carcinogen is a compound that induces local formation or growth of a tumor at the site of administration. Exemplary carcinogens that can be used include azoxymethane (AOM) and dextran sodium sulfate (DSS). Additional carcinogens can be selected by one of skill in the art, for example, depending on the subject and type of tumor to be induced.

In some examples, the cargo is formulated in an oncogenic nanoparticle delivery system. In some examples, the nanoparticle structure is formed spontaneously from polymers in aqueous solution. The polymers used to produce nanoparticles include, for example, poly (acrylamide), poly (ester), poly (alkylcyanoacrylates), poly (lactic acid) (PLA), poly (glycolic acids) (PGA), and poly (D,L-lactic-co-glycolic acid) (PLGA). The nanoparticles may have an average diameter of from 50 to 1000 nm and can be taken up into cells. Nanoparticles release their cargo as a function of their degradation. In some examples, the nanoparticle includes a cationic polymer that can self-assemble with a negatively charged expression vector (e.g., a viral vector or an expression vector) to form nanoparticles having a diameter of from 100 to 1000 nm. In one example, the nanoparticle is a liposome. The liposome is typically a lipid vesicle of one or more concentric phospholipid bilayers. In some cases, the phospholipids are composed of a hydrophilic head group and two hydrophobic chains to enable encapsulation of both hydrophobic and hydrophilic compounds. Liposomes for use as delivery systems have been described (see, for example, Paszko and Senge, Curr Med Chem 19(31)5239-5277, 2012; Immordino et al., Int J Nanomedicine 1(3):297-315, 2006; U.S. Patent Application Publication Nos. 2011/0268655; 2010/00329981).

### Methods of Use

Provided herein are methods of using the disclosed patches, for example, to deliver one or more cargoes that promote or induce tumor formation or growth, or alternatively, to deliver a therapeutic agent or candidate therapeutic agent. As discussed above, one use of the patches is to produce animal tumor models that more closely mimic human disease, for example for use in pre-clinical models of cancer therapies. In other examples, the disclosed patches can be used to deliver a therapeutic agent, either in a pre-clinical animal model or in a human or veterinary subject. In one example, the method includes delivering a patch including a cargo to a predetermined location of a tissue (such as an inner lumen of a target tissue); adhering the base layer of the patch to the predetermined location of the tissue; and releasing the cargo from the delivered patch into the predetermined location of the tissue to which it is adhered over a period of time in order to induce formation or growth of a tumor at the predetermined location of the tissue.

A patch comprising a hydrogel with a base layer (e.g., a polydopamine or fibrin base layer), such as one of the hydrogel patches described above, can be flexible, thereby allowing various manipulations of the patch such as repeated folding and un-folding.

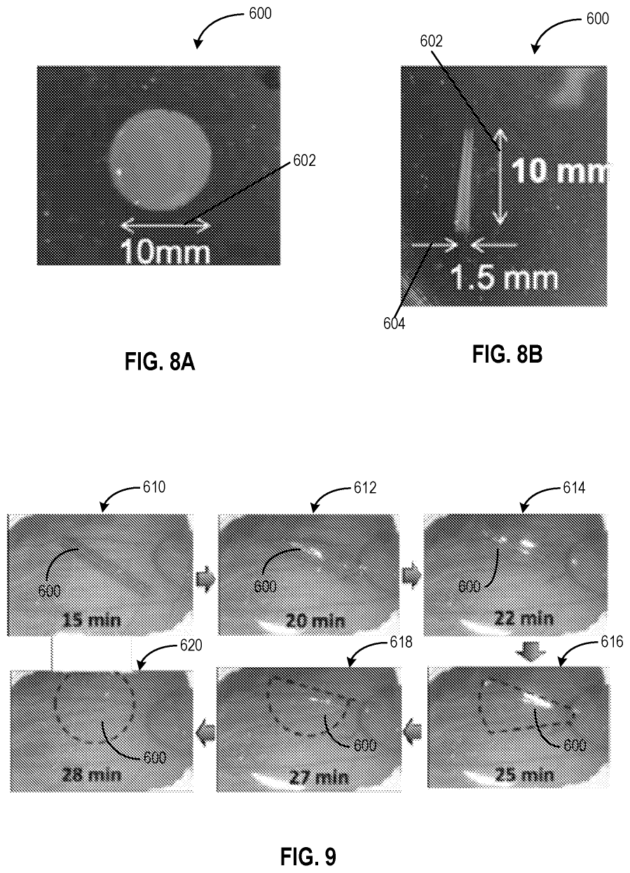

FIGS. 8A-8B show an exemplary patch 600 comprising a hydrogel including a plurality of microchannels and a base layer, the patch configured as a disc having a diameter 602 of approximately 10 mm (shown in the front view of FIG. 8A) and a thickness 604 of approximately 1.5 mm (shown in the side view of FIG. 8B). It should be noted that the dimensions of the patch 600 are exemplary, and other dimensions of the patch are possible, as described herein.

FIG. 9 shows the patch 600 in various stages of unfolding, thereby demonstrating its ability to be transformed into a tubular shape (e.g., for transport and/or delivery to a target tissue) and then undergo spontaneous (by itself, without assistance) or external force-induced unrolling (e.g., via an external device such as biopsy forceps). At 610, the patch is rolled into a tube which, in some examples, can be loaded into a delivery apparatus, such as an endoscope (as described further below). After delivery at an implantation site (target tissue or predetermined location of the tissue), the patch 600 can begin to unroll, as shown at 612. Further stages of unrolling of the patch 600 are shown at 614, 616, and 618, until the patch 600 is in a fully unfolded state at 620. FIG. 9 shows exemplary time periods for the various stages of unrolling of the patch 600 (over a time period of about 30 minutes from 610 to 620). However, these time periods are exemplary and may change based on a size and composition of the patch.

Thus, due to its flexible nature, it can be possible to deliver the patch to a target tissue through a delivery apparatus, such as an endoscope, catheter, or needle. Such delivery apparatuses provide for less invasive placement of a patch at a target tissue (as compared to surgical transplantation of tumor cells). In some examples, the patch can be delivered to an external surface of a solid organ or percutaneously into an interior of a solid organ via a needle (e.g., by placing the patch inside the needle and pushing it out of the needle with a stylet upon reaching an implantation site).

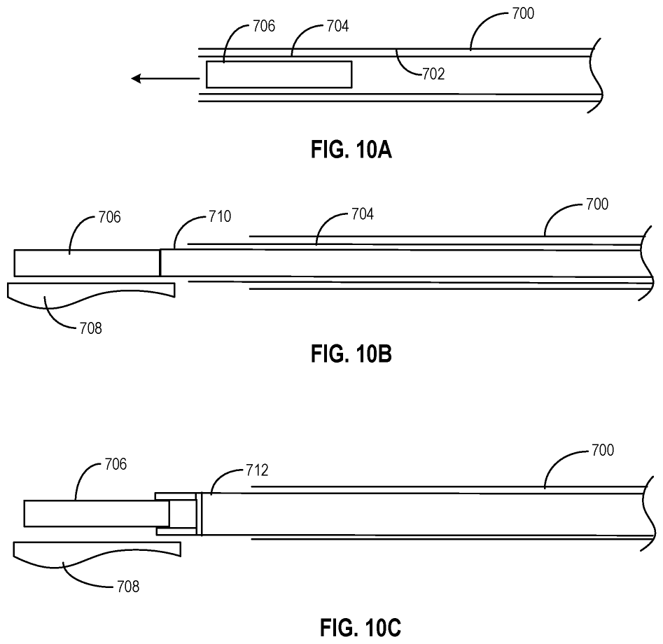

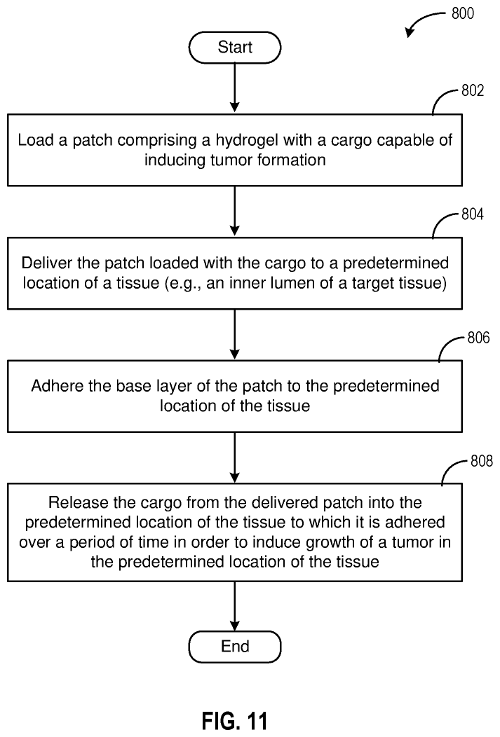

An exemplary method for delivering a patch to a target tissue using an endoscope is shown in FIGS. 10A-10C. Further, an exemplary method 800 for inducing growth of a tumor in a target tissue (or producing a somatic-cell tumor at a predetermined location of a tissue) using a patch is presented at FIG. 11. In the description of method 800 below, reference is made to FIGS. 10A-10C, as one possible example of delivering the patch to the target tissue. However, other delivery methods are possible, such as using a needle to deliver the patch to an outer surface or interior of a solid organ.

Method 800 begins at 802, which includes loading a patch comprising a hydrogel with a cargo. The patch can be any one of the patches described herein and is shown as patch 706 in the example presented at FIGS. 10A-10C, as described further below. For example, the patch can comprise a plurality of microchannels within a body of the hydrogel (or cargo layer of the patch) and the base layer disposed at a base of the body. Thus, the loading at 802 can include loading the plurality of microchannels of the patch with any of the cargo described above (such as via method 400 of FIG. 6 or method 500 of FIG. 7). In other examples, the method utilizes a patch that is pre-loaded with a cargo (for example, the method begins at 804, below).

The method 800 continues to 804, which includes delivering the patch to a predetermined location of a selected tissue. In some examples, the delivering at 804 can include delivering the patch to an inner lumen of a target tissue (such as shown in FIG. 1B). In other examples, the selected tissue can be a tissue of a solid organ and the delivery at 804 can include delivering the patch to an exterior surface of the selected tissue. In particular examples, the target tissue is bladder, esophagus, stomach, pancreas, intestine (such as colon), liver, lung, heart, kidney, bone, and the like. The selected tissue is present in a subject, such as a laboratory, veterinary, or human subject. In some examples, the subject is a mouse, rabbit, or pig. The subject may be a transgenic animal, for example, transgenic for one or more inducible genes. In one example, the transgenic animal is an oncopig. In other examples, the subject is a wild type animal (e.g., an animal that is not transgenic or otherwise genetically altered).

In some examples, the delivering at 804 can include folding or rolling the patch into a folded or rolled delivery state (e.g., such as the configuration shown at 610 in FIG. 9) and inserting it into an inner lumen of a delivery apparatus and then delivering the patch to the target tissue with the delivery apparatus. In some examples, the delivery apparatus can be an endoscope or other type of delivery catheter. In other examples, the delivery apparatus can be a syringe, needle, or other device that allows the patch to be delivered to a solid organ via percutaneous injection.

As illustrated in the example of FIG. 10A, an endoscope 700 can comprise an inner lumen 702 (or channel), defined by an inner wall of the endoscope 700 that runs an entire length of the endoscope 700. As such, a catheter 704 can be placed into the inner lumen 702. In some examples, the catheter 704 and endoscope 700 are coaxial with one another. A patch 706 (which can be any one of the patches described herein) can be folded or rolled up into the folded or rolled delivery state and then inserted into an inner lumen of the catheter 704, in its folded or rolled up state.

Once the endoscope (or other delivery apparatus) is navigated toward and nears the implantation site for the patch 706, such as the inner lumen of the target tissue 708, the catheter 704 can be extended toward the target tissue 708 and then the patch 706 can be pushed out of the catheter 704 (FIG. 10B). As one example, as shown in FIG. 10B, the patch 706 can be pushed out of the catheter 704 using a pushing element 710, such as a shaft. In some examples, if the target tissue 708 is an inner lumen of a luminal organ and the patch 706 can be pushed into the inner lumen of the target tissue 708.

In some examples, once the patch 706 is pushed out of the catheter 704 (FIG. 10B), the patch can be further moved or positioned into a desired position at the target tissue 708 (e.g., against a luminal wall of the target tissue 708) with a grabbing element 712, such as biopsy pinchers or forceps that can extend through the endoscope 700 and/or the catheter 704 (FIG. 10C). The grabbing element 712 can place the patch 706 into a desired position at the target tissue 708. The patch 706 can then spontaneously unfold or unroll against the target tissue 708 (e.g., against the inner lumen of the target tissue 708) or the grabbing element 712 can be used to initiate (via an applied pressure) or perform the unfolding or unrolling of the patch 706 against the target tissue 708. The endoscope 700 can then be removed from the implantation site and the subject.

Thus, the method at 804 can include positioning the patch at the predetermined location of the tissue. Clinically relevant tumors are located at luminal or parenchymal sites based on tissue anatomic structure. Also, in large organs, position of tumors is important with respect to the delivery or removal of such tumors by either surgery, radiation or directed energy. Hence, the patches and the delivery methods for such patches described herein enable the precise positioning of the patch (e.g., at the predetermined location of the tissue) to induce a given tumor in a given site at a defined time relevant to other co-morbidities.

Returning to the method 800 of FIG. 11, at 806, the method can include adhering the polydopamine base layer of the patch to the predetermined location of the tissue. For example, upon contact with the predetermined location of the tissue, the base layer of the patch (e.g., the base layer 204 shown in FIG. 2) can be activated and adhere to the tissue, thereby securing (effectively gluing) the patch to the tissue at the predetermined location. In some examples, the folded or rolled patch can be loaded into the catheter (catheter 704 of FIG. 10A) such that after being pushed out of the catheter, the base layer of the patch faces the tissue. In some examples, activation of the base layer occurs by exposure of the base layer to the tissue, for example by unfolding or unrolling of the patch.

After adhering to the predetermined location of the tissue (e.g., as shown in FIG. 5), the method 800 continues to 808 to release the cargo from the delivered patch into the predetermined location of the tissue to which it is adhered over a period of time in order to induce formation or growth of a tumor in the predetermined location of the tissue. For example, after contact with the tissue, the patch can begin to release its cargo controllably (e.g., at a predetermined diffusion rate that is based on a size and tortuosity of the microchannels and charge density of the hydrogel) and locally through the microchannels. By releasing its cargo controllably and substantially continuously over the period of time (e.g., 1-3 weeks or several minutes to several weeks), rather than all at once, the cargo can effectively cause cell division that is required for inducing tumor growth.

In some examples, the method at 808 can further include automatically degrading the patch at the target tissue over a predetermined length of time. For example, within a predetermined time period (e.g., 3-5 weeks), the patch can disappear by degradation of the hydrogel and polydopamine layer while causing little or no inflammation in the subject (e.g., at the target tissue). The patches described herein can be configured to minimize an immune response (e.g., inflammation) upon implantation at the target tissue. For example, by utilizing a biocompatible hydrogel for the patch and by containing the cargo within microchannels of the patch (which increase a surface area inside the patch), the patches described herein elicit little to no immune reaction at the implantation site.

### EXAMPLES

The following examples are provided to illustrate certain particular features and/or configurations. These examples should not be construed to limit the disclosure to the particular features or configurations described.

### Example 1-Ex Vivo Testing with a Patch

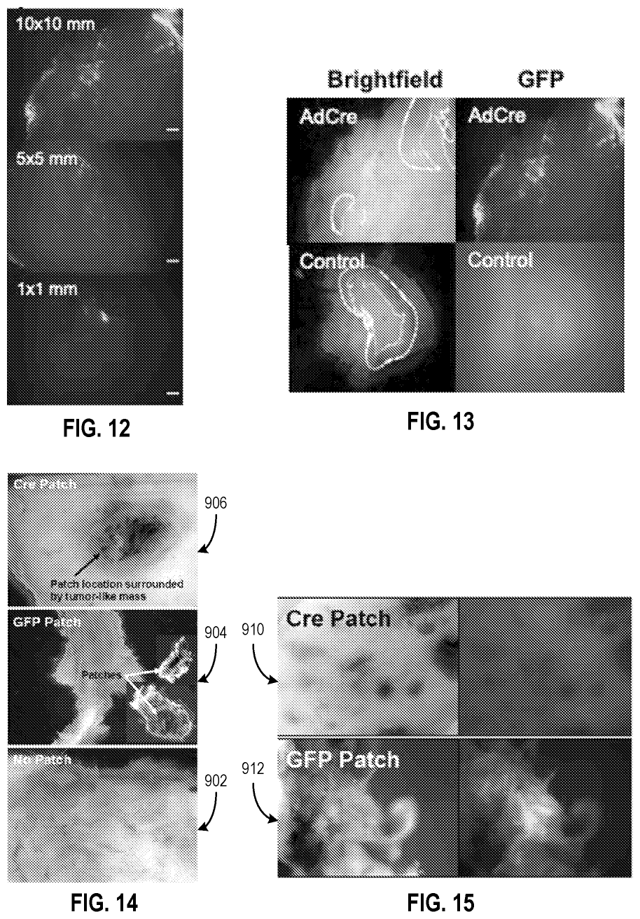

Patches loaded with AdCre as its cargo and ranging in size from 1×1 mm, 5×5 mm and 10×10 mm (FIG. 12) were secured on the bladder epithelium ex vivo and AdCre was effectively transferred to epithelial cells triggering the expression of Cre-GFP (green fluorescent protein) within 24 hours. FIG. 12 shows microscopic images of the three sized patches and GFP expression in each of the patches following placement at the bladder epithelium. Also, the induced expression of Cre-GFP was confined to the cells located under the patch and did not result in transduction of the surrounding bladder epithelial cells via diffusion, as shown in FIG. 13. For example, FIG. 13 presents a brightfield image of a tissue exposed to the AdCre via the patch and a control tissue, as well as showing Ad-GFP expression (shown by green fluorescence) in the tissue exposed to the patch and the control tissue.

### Example 2-In Vivo Tumor Induction with a Patch

Patches (referred to below as TIPs) were infused (loaded) with either AdCre or an adenoviral vector encoding GFP only (AdGFP) and placed into the subcutaneous space in the neck area between the ears of several Oncopigs (n=2). This location allowed for visual observation of tumor induction while limiting the ability of the Oncopig to touch or lick the incision site. Each Oncopig received a TIP on the left side which contained AdCre and a TIP loaded with AdGFP on the right side. Therefore, each animal served as its own control. Following seven days of TIP placement within the Oncopig, the formation of a tumor mass was observed on the left side of both Oncopigs (FIG. 14). FIG. 14 presents microscopic images that show tumor formation around the implanted TIPs (shown at 906). As shown in FIG. 14, no tumor formation occurred surrounding the control TIPs (shown at 904) or at the subcutaneous tissue regions without TIP exposure (shown at 902).

Animals were euthanized one week post tumor induction and the tumor mass was dissected as was the right side incision site to permit analysis of TIP survival and whether GFP activity could be observed. As shown in the microscopic image 906 in FIG. 14, TIPs loaded with AdCre induced a tumor mass within seven days in vivo. TIPs were identified within the tumor masses indicating that the TIP was still intact and had not biodegraded by seven days, and thus was still providing oncogenesis signals. At the control TIP site (TIPs loaded with AdGFP), as shown in the microscopic image 904 in FIG. 14, the TIPs were still evident but no tumor-like mass developed around the TIP. No foreign body inflammation or nodule formation was observed.

The resulting tumors and control TIP sites were subjected to fluorescent microscopy within three hours of tissue capture to assess GFP expression. As shown in FIG. 15, GFP activity was observed in the tumors induced by TIPs carrying AdCre (shown at 910) and subcutaneous tissue surrounding the control TIPs carrying AdGFP (shown at 912). This provides evidence that the TIP features support the sustained release of functional adenoviral vectors in vivo and that these vectors are not immediately diffusing out into the tissue sites but being contained and protected from tissue enzyme-induced degradative processes. Hence, TIPs would support longer term tumor induction in vivo. When such experiments were conducted in vitro, cells exposed to AdCre or AdGFP began expressing GFP within 24 hours, with fluorescence returning to background levels within 3-14 days.

Hence, the TIP enables a defined release of adenoviral vectors and delays immediate cellular uptake of AdCre. AdCre is capable of transducing all cells but only triggers oncogenesis in dividing cells (removal of STOP signal); hence being restricted to activating transgene expression in cells actively dividing at the time of AdCre injection. Because of this, local tissue proliferation and AdCre clearance rates affect the magnitude and efficacy of tumor induction when injecting AdCre directly into Oncopig tissues. However, the TIP as designed provides a continued release of AdCre and AdGFP to lengthen the exposure time, therefore extending the time course of induction.

In addition, patches (TIPs or TMPs) of varying sizes, shape, and microstructure have been developed that allow for up to 1,000 fold differences in viral vector loads (such as AdCre and AdGFP vector loads), as shown in Table 1. The ability to engineer patch geometry and adenoviral vector loads further demonstrates that the patches described herein can provide a defined signal over a defined time period.

TABLE 1
Exemplary patch dimensions, coverage, and vector delivery amounts

| | | | | | | | Patch | | | |
|---|---|---|---|---|---|---|---|---|---|---|
| | | | | | | | Coverage | Viral Vector Delivery Amounts | | |
| | | | | | | | (cell | (example conc = 1 × 10\hat{ }8 PFU/μl) | | |
| | Patch Dimensions | | | | | | area = | Vol | | Multiplicity |
| | | | | | | Loading | 200 μm²) | Viral | Vector/ | of |
| TIP/ | Length | Width | Thickness | Area | Volume | Volume | Cells/ | Vector | Patch | Infection |
| TMP | (mm) | (mm) | (mm) | (mm²) | (mm³) | (μl) | Patch | (μl) | (PFU) | (PFU/cell) |
| Large | 10 | 10 | 1 | 100 | 100 | 200 | 5 × 10\hat{ }5 | 200 | 2 × 10\hat{ }10 | 40,000 |
| | | | | | | | | 20 | 2 × 10\hat{ }9 | 4,000 |
| | | | | | | | | 2 | 2 × 10\hat{ }8 | 400 |
| Medium | 5 | 5 | 1 | 25 | 25 | 50 | 1.3 × 10\hat{ }5 | 50 | 5 × 10\hat{ }9 | 40,000 |
| | | | | | | | | 5 | 5 × 10\hat{ }8 | 4,000 |
| | | | | | | | | 0.5 | 5 × 10\hat{ }7 | 400 |
| Small | 1 | 1 | 1 | 1 | 1 | 2 | 5 × 10\hat{ }3 | 2 | 2 × 10\hat{ }8 | 40,000 |
| | | | | | | | | 0.2 | 2 × 10\hat{ }7 | 4,000 |
| | | | | | | | | 0.02 | 2 × 10\hat{ }6 | 400 |

### Example 3-Development of Oncopig Lung Adenocarcinoma Cell Lines

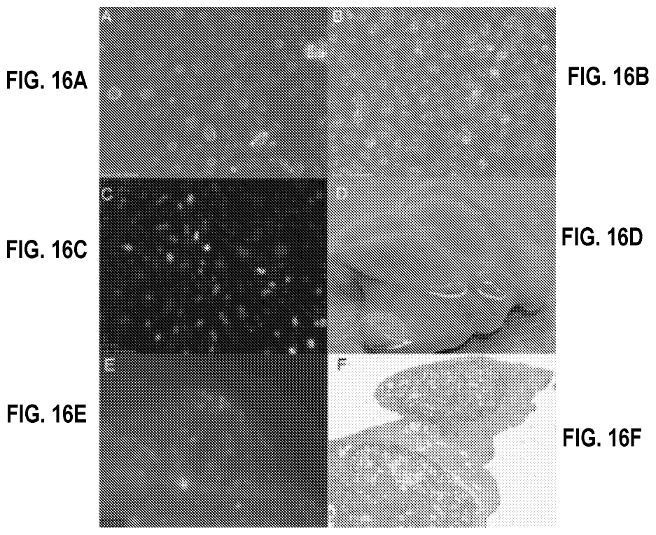

In order to develop Oncopig lung adenocarcinoma cell lines, type II pneumocytes were isolated from Oncopig lung samples and exposed to AdCre (FIG. 16A). Consistent with findings in humans, expression of the KRASG12D mutant protein induced vacuolization and cell death (FIG. 16B). In order to avoid this induced cell death, Oncopig type II pneumocytes were transduced with a lentiviral vector encoding Cre recombinase and a CDK4R24L expression vector under control of a type II pneumocyte-specific promoter, resulting in cellular proliferation and positive staining with clinically relevant lung adenocarcinoma diagnostic markers (FIG. 16C). Delivery of this lentiviral vector to Oncopig lung segments ex vivo resulted in lung masses containing GFP expressing cells and histological structures consistent with lung adenocarcinoma (FIG. 16D-16F). These tumor masses also stained positive ki-67 (indicating increased proliferation) and the KRASG12D transgene. The results of this study demonstrate the ability to induce oncogenesis in Oncopig cells through a combination of gene editing and transgene activation.

### Example 4-Development of Rabbit HCC Cell Lines

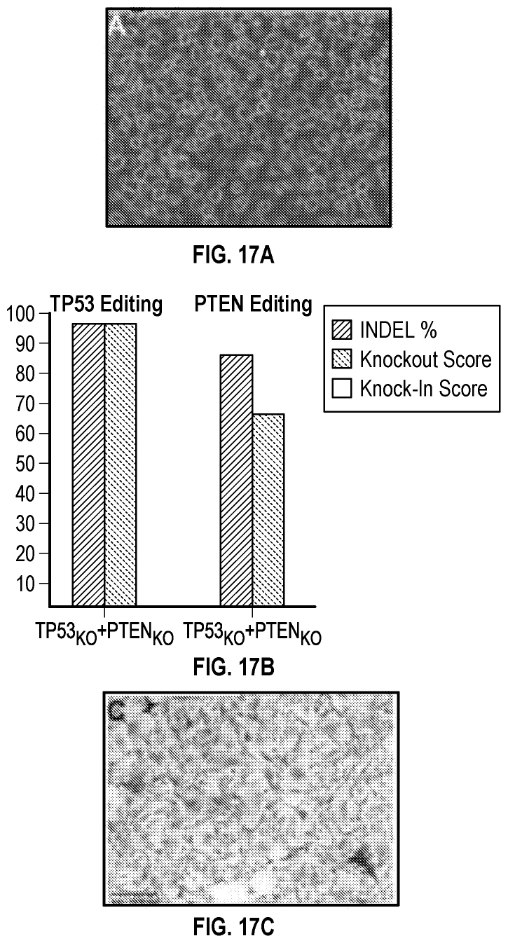

In order to demonstrate the ability to induce oncogenesis solely through gene editing in wild type animals, isolated rabbit hepatocytes were exposed to CRISPR-Cas9 components targeting TP53 and PTEN. Transfection of isolated hepatocytes resulted in TP53 and PTEN KO as confirmed via sequencing (FIGS. 17A and 17B). While primary hepatocytes quickly died out following 2 weeks of culturing, TP53 and PTEN KO cell lines proliferated and have persisted in culture for over 4 months. These cells stain positive for the hepatocyte specific marker arginase-1, thus confirming their hepatocellular origin (FIG. 17C). Together these results confirm the ability to utilize gene editing techniques to induce oncogenesis in primary, wild type cells.

### Example 5-Method of Making a Hydrogel Patch

Alginate (Mw≈250 000 g mol-1, FMC Biopolymer) sterilized via filtration was dissolved in 0.1 M 2-(N-morpholino) ethanesulfonic acid (MES) buffer at a concentration of 2% (w/v). The alginate solution was sequentially mixed with microparticles (if necessary), sulfonated N-hydroxysuccimide (Sulfo-NHS; Thermo Scientific), adipic acid dihydrazide (AAD; Sigma-Aldrich), and 1-ethyl-3-(3-dimethylaminopropyl) carbodiimide (EDC; Thermo Scientific). The pregelled mixture was cured in a space between two glass plates separated by 1 mm spacer. Then, hydrogel sheets were incubated in DI water at room temperature for 12 h. Then, to prepare the microchanneled hydrogel, the gel was placed on top of a copper plate with controlled temperatures. All gels were surrounded by styrene foam for insulation. The frozen sample was freeze-dried to introduce microchannels through the alginate gel disk. Finally, the dehydrated, microchanneled matrix was rehydrated by dropping aqueous media or oncogene suspension on the top of the sample. In contrast, the microporous gel was prepared via sequential freezing of the alginate gel in a copper container, lyophilization, and rehydration.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A patch, comprising: a base layer configured to adhere to a predetermined location of a tissue; and a hydrogel layer comprising a plurality of microchannels that are configured to store and release a gene-modifying vector over a time period and in a quantity sufficient to produce a somatic cell tumor through manipulation of the genome of somatic cells at the predetermined location.

Example 2. The patch of any example herein, particularly example 1, wherein the patch is configured to continually release the gene-modifying vector from the plurality of microchannels over the time period when the patch is positioned at the predetermined location of the tissue.

Example 3. The patch of any example herein, particularly either example 1 or example 2, wherein the patch is configured to degrade after three to five weeks of contact with the tissue.

Example 4. The patch of any example herein, particularly any one of examples 1-3, wherein the hydrogel layer further comprises the gene-modifying vector, and wherein the gene-modifying vector comprises an oncogenic viral vector disposed within lumina of the microchannels.

Example 5. The patch of any example herein, particularly any one of examples 1-3, wherein the hydrogel layer further comprises the gene-modifying vector, and wherein the gene-modifying vector comprises an adenoviral vector expressing Cre recombinase (AdCre) disposed within lumina of the microchannels.

Example 6. The patch of any example herein, particularly either example 4 or example 5, wherein the gene-modifying vector is carried within oncogenic nanoparticles disposed within lumina of the microchannels.

Example 7. The patch of any example herein, particularly any one of examples 1-6, wherein the microchannels of the plurality of microchannels are anisotropically aligned with one another in a hydrogel of the hydrogel layer.

Example 8. The patch of any example herein, particularly any one of examples 1-7, wherein the cargo layer of the patch comprises an alginate and divalent hydrazide group cross-linked hydrogel.

Example 9. The patch of any example herein, particularly any one of examples 1-8, wherein a diameter of the plurality of microchannels is in a range of 50-1000 μm.

Example 10. The patch of any example herein, particularly any one of examples 1-9, wherein a thickness of the patch is in a range of 0.05-1.5 mm, wherein a length of the patch is in a range of 1-10 mm, and wherein a width of the patch is in a range of 1-10 mm.

Example 11. A method for the production of a somatic-cell tumor at a predetermined location of a tissue, comprising: positioning, at the predetermined location, a patch comprising a base layer configured to adhere to the predetermined location and a hydrogel layer comprising a plurality of microchannels containing a releasable oncogenic cargo stored within the plurality of microchannels, such that the oncogenic cargo is released from the hydrogel layer and produces a somatic-cell tumor at the predetermined location through manipulation of the genome of the somatic cells.

Example 12. The method of any example herein, particularly example 11, wherein the predetermined location is a luminal surface of the tissue.

Example 13. The method of any example herein, particularly example 12, wherein the tissue is a tissue of one of a pancreas, bladder, colon, rectum, esophagus, stomach, or throat.

Example 14. The method of any example herein, particularly example 11, wherein the predetermined location is an exterior surface of the tissue.

Example 15. The method of any example herein, particularly example 14, wherein the tissue is one of a neuron, brain, or ovary.

Example 16. The method of any example herein, particularly any one of examples 11-15, wherein the patch is configured to flex and be positioned against one of a luminal surface or exterior surface of the tissue.

Example 17. The method of any example herein, particularly any one of examples 11-16, wherein positioning the patch at the predetermined location of the tissue includes adhering the base layer of the patch to a luminal surface or exterior surface of the tissue.

Example 18. The method of any example herein, particularly example 17, wherein the base layer comprises one of a fibrin or polydopamine glue.

Example 19. The method of any example herein, particularly any one of examples 11-18, further comprising, prior to the positioning the patch at the predetermined location of the tissue, folding the patch into a folded delivery state and inserting it into an inner lumen of a delivery apparatus, and further comprising navigating the delivery apparatus toward the predetermined location of the tissue.

Example 20. The method of any example herein, particularly example 19, wherein the delivery apparatus is an endoscope.

Example 21. The method of any example herein, particularly example 19, wherein the delivery apparatus is a needle.

Example 22. The method of any example herein, particularly any one of examples 19-21, wherein positioning the patch at the predetermined location of the tissue includes pushing the folded patch out of a distal end portion of the delivery apparatus at the predetermined location of the tissue and unfolding the patch from the folded delivery state to an unfolded state such that the base layer is positioned against a surface of the predetermined location of the tissue.

Example 23. The method of any example herein, particularly any one of examples 11-22, further comprising, after positioning the patch at the predetermined location of the tissue, automatically degrading the patch at the predetermined location of the tissue over a predetermined length of time.

Example 24. The method of any example herein, particularly example 23, wherein the predetermined length of time is in a range of three to five weeks.

Example 25. The method of any example herein, particularly any one of examples 11-24, wherein the oncogenic cargo comprises a gene-modifying vector.

Example 26. The method of any example herein, particularly any one of examples 11-24, wherein the oncogenic cargo comprises a nucleic acid comprising CRISPR components for introducing one or more tumor driver mutations into the somatic cells of the tissue.

Example 27. The method of any example herein, particularly any one of examples 11-24, wherein the oncogenic cargo is carried within nanoparticles stored within the plurality of microchannels of the hydrogel layer of the patch.

Example 28. The method of any example herein, particularly any one of examples 11-24, wherein the oncogenic cargo comprises one or more of carcinogens, cell-division factors, or angiogenic factors.

Example 29. The method of any example herein, particularly any one of examples 11-27, wherein the hydrogel layer of the patch further comprises an auxiliary cargo stored within the plurality of microchannels, and further comprising releasing the auxiliary cargo from the hydrogel layer in order to modulate one or more of a biology, microenvironment, or growth rate of the somatic-cell tumor produced at the predetermined location of the tissue.

Example 30. The method of any example herein, particularly example 29, wherein the auxiliary cargo comprises carcinogens.

Example 31. The method of any example herein, particularly either example 29 or example 30, wherein the auxiliary cargo comprises one or more of cell-division factors and angiogenic factors.

Example 32. The method of any example herein, particularly any one of examples 11-31, wherein the tissue is in a subject, and wherein the subject is a transgenic or a wild type animal.

Example 33. A method of making a microchannel-containing hydrogel patch, comprising: forming a hydrogel by cross-linking gel-forming molecules in a cross-linking solution; adhering a base layer on a surface of the hydrogel, the base layer configured to adhere to a tissue; lyophilizing the hydrogel under conditions sufficient to generate a plurality of microchannels such that a cryogel with the plurality of microchannels is formed; and rehydrating the cryogel in a solution containing a selected cargo.

Example 34. The method of any example herein, particularly example 33, wherein the selected cargo is an oncogenic viral vector.

Example 35. The method of any example herein, particularly example 33, wherein the selected cargo is an adenoviral vector expressing Cre recombinase (AdCre).

Example 36. The method of any example herein, particularly example 33, wherein the selected cargo is oncogenic nanoparticles carrying cancer causing material.

Example 37. The method of any example herein, particularly example 33, wherein the selected cargo is carcinogens.

Example 38. The method of any example herein, particularly any one of examples 33-37, wherein the generated microchannels of the plurality of microchannels are anisotropically aligned with one another in the hydrogel.

Example 39. The method of any example herein, particularly any one of examples 33-38, wherein the hydrogel is an alginate and divalent hydrazide group cross-linked hydrogel.

Example 40. The method of any example herein, particularly any one of examples 33-39, wherein a diameter of the microchannels of the plurality of microchannels is in a range of 50-1000 μm.

Example 41. The method of any example herein, particularly any one of examples 33-40, wherein a thickness of the patch is in a range of 0.05-1.5 mm, wherein a length of the patch is in a range of 1-10 mm, and wherein a width of the patch is in a range of 1-10 mm.

Example 42. A method of making a microchannel-containing hydrogel patch, comprising: mixing a flexible cross-linked hydrogel with a solution comprising a cargo; lyophilizing the flexible cross-linked hydrogel and cargo under conditions sufficient to generate a plurality of microchannels comprising the cargo in the hydrogel; and adhering a base layer on a surface of the hydrogel, the base layer configured to adhere to a tissue.

Example 43. The method of any example herein, particularly example 42, wherein the cargo is an oncogenic viral vector.

Example 44. The method of any example herein, particularly example 42, wherein the cargo is an adenoviral vector expressing Cre recombinase (AdCre).

Example 45. The method of any example herein, particularly example 42, wherein the cargo is oncogenic nanoparticles carrying cancer causing material.

Example 46. The method of any example herein, particularly example 42, wherein the cargo is carcinogens.

Example 47. The method of any example herein, particularly any one of examples 42-46, wherein the generated microchannels of the plurality of microchannels are anisotropically aligned with one another in the hydrogel.

Example 48. The method of any example herein, particularly any one of examples 42-47, wherein the hydrogel is an alginate and divalent hydrazide group cross-linked hydrogel.

Example 49. The method of any example herein, particularly any one of examples 42-48, wherein a diameter of the microchannels of the plurality of microchannels is in a range of 50-1000 μm.

Example 50. The method of any example herein, particularly any one of examples 42-49, wherein a thickness of the patch is in a range of 0.05-1.5 mm, wherein a length of the patch is in a range of 1-10 mm, and wherein a width of the patch is in a range of 1-10 mm.

In view of the many possible examples to which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A patch, comprising:
a base layer configured to adhere to a predetermined location of a tissue; and
a hydrogel layer comprising a plurality of microchannels that are configured to store and release a gene-modifying vector over a time period and in a quantity sufficient to produce a somatic cell tumor through manipulation of the genome of somatic cells at the predetermined location.

2. (canceled)

3. (canceled)

4. The patch of claim 1, wherein the hydrogel layer further comprises the gene-modifying vector, and wherein the gene-modifying vector comprises an oncogenic viral vector disposed within lumina of the microchannels.

5. The patch of claim 1, wherein the hydrogel layer further comprises the gene-modifying vector, and wherein the gene-modifying vector comprises an adenoviral vector expressing Cre recombinase (AdCre) disposed within lumina of the microchannels.

6. The patch of claim 4, wherein the gene-modifying vector is carried within oncogenic nanoparticles disposed within lumina of the microchannels.

7. The patch of claim 1, wherein the microchannels of the plurality of microchannels are anisotropically aligned with one another in a hydrogel of the hydrogel layer.

8. The patch of claim 1, wherein the cargo layer of the patch comprises an alginate and divalent hydrazide group cross-linked hydrogel.

9. The patch of claim 1, wherein a diameter of the plurality of microchannels is in a range of 50-1000 μm.

10. The patch of claim 1, wherein a thickness of the patch is in a range of 0.05-1.5 mm, wherein a length of the patch is in a range of 1-10 mm, and wherein a width of the patch is in a range of 1-10 mm.

11. A method for the production of a somatic-cell tumor at a predetermined location of a tissue, comprising:
positioning, at the predetermined location, a patch comprising a base layer configured to adhere to the predetermined location and a hydrogel layer comprising a plurality of microchannels containing a releasable oncogenic cargo stored within the plurality of microchannels, such that the oncogenic cargo is released from the hydrogel layer and produces a somatic-cell tumor at the predetermined location through manipulation of the genome of the somatic cells.

12.-15. (canceled)

16. The method of claim 11, wherein the patch is configured to flex and be positioned against one of a luminal surface or exterior surface of the tissue.

17. The method of claim 11, wherein positioning the patch at the predetermined location of the tissue includes adhering the base layer of the patch to a luminal surface or exterior surface of the tissue.

18. (canceled)

19. The method of claim 11, further comprising, prior to the positioning the patch at the predetermined location of the tissue, folding the patch into a folded delivery state and inserting it into an inner lumen of a delivery apparatus, and further comprising navigating the delivery apparatus toward the predetermined location of the tissue.

20. (canceled)

21. (canceled)

22. The method of claim 19, wherein positioning the patch at the predetermined location of the tissue includes pushing the folded patch out of a distal end portion of the delivery apparatus at the predetermined location of the tissue and unfolding the patch from the folded delivery state to an unfolded state such that the base layer is positioned against a surface of the predetermined location of the tissue.

23. The method of claim 11, further comprising, after positioning the patch at the predetermined location of the tissue, automatically degrading the patch at the predetermined location of the tissue over a predetermined length of time.

24. The method of claim 23, wherein the predetermined length of time is in a range of three to five weeks.

25.-32. (canceled)

33. A method of making the patch of claim 1, comprising:
forming a hydrogel by cross-linking gel-forming molecules in a cross-linking solution;
adhering a base layer on a surface of the hydrogel, the base layer configured to adhere to a tissue;
lyophilizing the hydrogel under conditions sufficient to generate a plurality of microchannels such that a cryogel with the plurality of microchannels is formed; and
rehydrating the cryogel in a solution containing a selected cargo.

34. The method of claim 33, wherein the selected cargo is an oncogenic viral vector.

35.-37. (canceled)

38. The method of claim 33, wherein the generated microchannels of the plurality of microchannels are anisotropically aligned with one another in the hydrogel.

39.-41. (canceled)

42. A method of making the patch of claim 1, comprising:
mixing a flexible cross-linked hydrogel with a solution comprising a cargo;
lyophilizing the flexible cross-linked hydrogel and cargo under conditions sufficient to generate a plurality of microchannels comprising the cargo in the hydrogel; and
adhering a base layer on a surface of the hydrogel, the base layer configured to adhere to a tissue.

43.-47. (canceled)

48. The method of claim 42, wherein the hydrogel is an alginate and divalent hydrazide group cross-linked hydrogel.

49. (canceled)

50. (canceled)
